(19) United States
(12) Patent Application Publication
(10) Pub. No.: US 2025/0241969 A1
(43) Pub. Date: Jul. 31, 2025

(54) NEOADJUVANT CANCER TREATMENT

(71) Applicant: Duke University, Durham, NC (US)

(72) Inventors: Darell Bigner, Durham, NC (US); Matthias Gromeier, Durham, NC (US); Smita Nair, Durham, NC (US); Henry Friedman, Durham, NC (US); Annick Desjardins, Durham, NC (US)

(21) Appl. No.: 19/068,967

(22) Filed: Mar. 3, 2025

Related U.S. Application Data
(63) Continuation of application No. 17/044,645, filed on Oct. 1, 2020, now abandoned.
(60) Provisional application No. 62/823,277, filed on Mar. 25, 2019.
(60) Provisional application No. 62/651,470, filed on Apr. 2, 2018.

(51) Int. Cl. A61K 35/768; C07K 16/28

(52) U.S. Cl. CPC A61K 35/768; C07K 16/2818; C07K 16/2827

(57) ABSTRACT

Provided is a method of treating a tumor in an individual by neoadjuvant therapy, wherein the individual has not previously undergone treatment to effectively reduce tumor burden, the method comprising administering an oncolytic chimeric poliovirus construct, or an oncolytic chimeric poliovirus construct and an immune checkpoint inhibitor, followed by reduction of the tumor. The method may further comprise administration of immune checkpoint inhibitor or oncolytic chimeric poliovirus construct following reduction of tumor. Kits for performing the methods are also provided.

### CROSS-REFERENCE TO RELATED APPLICATIONS

This patent application claims the benefit of priority of U.S. Provisional Patent Application No. 62/651,470, filed Apr. 2, 2018, and U.S. Provisional Patent Application No. 62/823,277, filed Mar. 25, 2019, both of which are incorporated herein by reference in their entirety.

### STATEMENT REGARDING FEDERALLY SPONSORED RESEARCH

This invention was made with Government Support under Federal Grant No. R35-CA197264 awarded by the NCI/NIH and Federal Grant No. BC151083 awarded by the Department of Defense Breast Cancer Research Program Level 3 Breakthrough Award. The Federal Government has certain rights to this invention.

### TECHNICAL FIELD OF THE INVENTION

This invention is related to the area of anti-tumor therapy. In particular, it relates to oncolytic virus anti-tumor treatment in a neoadjuvant therapy.

### BACKGROUND OF THE INVENTION

PVSRIPO is a recombinant oncolytic poliovirus. It consists of the live attenuated type 1 (Sabin) PV vaccine containing a foreign internal ribosomal entry site (IRES) of human rhinovirus type 2 (HRV2). See Gromeier et al., PNAS 93:2370-2375 (1996) and U.S. Pat. No. 6,264,940. The IRES is a cis-acting genetic element located in the 5′ untranslated region of the poliovirus genome, mediating viral, m⁷G-cap-independent translation. The anti-tumor effects of PVSRIPO comprise direct, virus-mediated tumor cell killing; and secondary, host-mediated immune response directed against the tumor. See Brown et al., Sci Transl Med (: 4220 (2017). The virus has shown exciting and unexpected efficacy in humans. Nonetheless, there is a continuing need in the art to identify and develop anti-cancer treatments that provide one or more improved therapeutic benefits to humans, particularly for individuals with hard-to-treat cancers.

### SUMMARY OF THE INVENTION

According to one aspect of the invention, a method of treating a tumor in an individual by neoadjuvant therapy is provided. In this method, the individual has not previously undergone a treatment to reduce the tumor burden (e.g., no surgical treatment or radiation treatment to reduce tumor burden). An immune checkpoint inhibitor is also administered to the individual, either at the same time or sequentially in relation to (before or after administration of) a oncolytic chimeric poliovirus construct. After treatment with a therapeutically effective amount of oncolytic chimeric poliovirus construct and a therapeutically effective amount of an immune checkpoint inhibitor, the individual is then treated to reduce tumor burden. In one aspect, the oncolytic chimeric poliovirus construct, administered to the individual, comprises a Sabin type I strain of poliovirus with a human rhinovirus 2 (HRV2) internal ribosome entry site (IRES) in the poliovirus' 5′ untranslated region between the poliovirus' cloverleaf and said poliovirus' open reading frame.

According to another aspect of the invention a method of treating a tumor in an individual by neoadjuvant therapy is provided. In this method, the individual has not previously undergone a resection to treat the tumor (e.g., no surgical treatment to reduce tumor burden). An immune checkpoint inhibitor is administered to the individual. A oncolytic chimeric poliovirus construct is also administered to the individual, wherein the oncolytic chimeric poliovirus construct comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame (PVSRIPO). Subsequent to administration of the neoadjuvant therapy comprising immune checkpoint inhibitor and oncolytic chimeric poliovirus, the individual is treated to reduce tumor burden comprising surgical resection of the tumor. Such resection of tumor can occur in a time period ranging from 1 week to a month following administration of an immune checkpoint inhibitor and the oncolytic chimeric poliovirus.

According to further aspect of the invention, any one of the methods of neoadjuvant therapy described herein may further comprise administering a poliovirus immunization booster (e.g., trivalent inactivated IPOL from Sanofi-Pasteur) between 6 months and 1 week prior to administering the oncolytic chimeric poliovirus construct.

According to another aspect of the invention, any one of the methods described herein may further comprise adjuvant therapy following resection of the tumor, wherein such therapy comprises administering one or more of the oncolytic chimeric poliovirus construct or the immune checkpoint point inhibitor to the individual having tumor burden reduced. For example, following tumor resection or radiation treatment of tumor, an immune checkpoint inhibitor may be administered to the individual as needed in maintenance therapy. In another example, if tumor recurs following resection or radiation, oncolytic chimeric poliovirus may be administered to the individual.

According to a further aspect of the invention, provided is neoadjuvant therapy of a tumor in an individual, and use of oncolytic chimeric poliovirus construct by itself or in combination with an immune checkpoint inhibitor as a medicament or as compositions in neoadjuvant therapy of tumor, wherein the individual has not previously undergone a resection to treat the tumor, wherein the oncolytic chimeric poliovirus construct comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame; and wherein after the tumor is treated with a therapeutically effective amount of the oncolytic chimeric poliovirus construct, or a combination comprising a oncolytic chimeric poliovirus construct and a therapeutically effective amount of the immune checkpoint inhibitor, tumor burden is then reduced. The neoadjuvant therapy may further comprise one or more treatments, subsequent to reduction of tumor burden, comprising administering a therapeutically effective amount of the oncolytic chimeric poliovirus construct, or a therapeutically effective amount of an immune checkpoint inhibitor, or a combination thereof.

Also provided is a method for neoadjuvant immunotherapy of cancer comprising:
a) administering one or more immunotherapeutic agents in a therapeutically effective amount to an individual having tumor, wherein the one or more immunotherapeutic agents comprise a oncolytic chimeric poliovirus construct, or a oncolytic chimeric poliovirus construct and an immune checkpoint inhibitor administered sequentially in combination therapy; b) subsequent to receiving the one or more immunotherapeutic agents, treating the individual with anti-cancer therapy selected from the group consisting of surgery, radiation therapy, and a combination thereof, effective to reduce tumor burden (e.g., the amount of tumor) in the individual (i.e., the one or more immunotherapeutic agents is administered before the anti-cancer therapy). The oncolytic chimeric poliovirus construct or immune checkpoint inhibitor, or a combination thereof, may further comprise addition of a pharmaceutically acceptable carrier. In one aspect, the oncolytic chimeric poliovirus construct is PVSRIPO.

Provided is neoadjuvant therapy of tumor in an individual comprising administering an immune checkpoint inhibitor and a oncolytic chimeric poliovirus construct, each in a therapeutically effective amount, to the individual whose tumor has not previously undergone reduction by resection or radiation treatment, wherein the oncolytic chimeric poliovirus construct comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame; wherein after the tumor is treated with the oncolytic chimeric poliovirus construct and the immune checkpoint inhibitor, the tumor is then treated to reduce tumor burden; and wherein the neoadjuvant therapy provides an improved therapeutic benefit, as compared to adjuvant therapy using a combination of the oncolytic chimeric poliovirus construct and the immune checkpoint inhibitor. A therapeutic benefit may comprise one or more of: reduced inflammation around the site of the tumor (prior to and/or after resection); improved overall survival; improved disease-free survival; decreased likelihood of recurrence (in the primary organ and/or distant recurrence); decreased incidence of metastatic disease; and an increased antitumor immune response; or an improvement in overall objective response rate using the appropriate response assessment criteria known to those skilled in the art and depending on the type of cancer treated (e.g., for lymphoma, see Cheson et al., 2014, J. Clin. Oncology32 (27): 3059-3067; for solid nonlymphoid tumors, Response Evaluation Criteria In Solid Tumors (RECIST). Regarding reduced inflammation, it was discovered that those individuals with tumor, and particularly brain tumor, who are treated with the oncolytic chimeric poliovirus construct and experienced minimal or easily controllable inflammation demonstrated a better (more effective and/or more durable) antitumor response as compared to individuals who were treated with the oncolytic chimeric poliovirus construct and experienced extensive or hard to manage inflammation.

These and other aspects which will be apparent to those of skill in the art upon reading the specification and provides the art with new therapeutic regimens for treating cancer.

### BRIEF DESCRIPTION OF THE DRAWINGS

FIG. 1 is a diagram depicting the genetic structure of oncolytic chimeric poliovirus construct PVSRIPO. The poliovirus 5′ untranslated region (UTR) contains an internal ribosome entry site (IRES) from human rhinovirus B in place of the native poliovirus sequence between the cloverleaf at the 5′ end of the poliovirus and the poliovirus' open reading frame.

FIG. 2 is a Kaplan-Meier curve of overall survival for historical controls (red line) as compared to individuals treated with the various doses of PVSRIPO (blue line; “PVSRIPO”) with the y-axis as overall survival (“Survival Probability”) and the x-axis as the number of months.

FIG. 3 shows results using four different tumor cell lines representing breast (SUM149 and MDA-MB231), melanoma (DM6), and prostate (LNCaP) cancers. Dendritic cells (DCs) were seeded in dishes. Supernatant from onco-lysate was added to DC cultures and incubated. Supernatant was then removed and DCs were washed. DNase I-treated peripheral blood mononuclear cells (PBMCs) were incubated at 37° C. Non-adherent cells were harvested and stimulated with DCs loaded with poliovirus-induced tumor lysate at a responder cell to stimulator DC ratio of 10:1 in the presence of IL-7 in CTL stimulation media. T cells were harvested on day 12-14, counted and used as effector T cells in a europium-release CTL assay. Autologous DCs transfected with relevant and irrelevant tumor antigen-encoding mRNA were used as control targets. For DC control targets, mRNA-electroporated target cells were harvested, washed to remove all traces of media and labeled with europium (Eu). Alternatively, original target cells (Sum149, MDAMB231, LNCaP, or DM6) were labeled with Eu. Ten thousand europium-labeled targets (T) and serial dilutions of effector cells (E) at varying E:T ratios were incubated in 96-well V-bottom plates. The plates were centrifuged for 3 minutes and incubated at 37° C. 50 μl of the supernatant was harvested and added to 150 μl of enhancement solution in 96-well flat-bottom plates and europium release was measured by time resolved fluorescence using the VICTOR3 Multilabel Counter (Perkin-Elmer). Specific cytotoxic activity was determined using the formula: % specific release=[(experimental release−spontaneous release)/(total release−spontaneous release)]×100. Spontaneous release of the target cells was less than 25% of total release by detergent. Spontaneous release of the target cells was determined by incubating the target cells in medium without T cells. All assays were done in triplicate, bars represent average % lysis and error bars denote standard error of the mean.

FIG. 4A-FIG. 4D show results of in vivo testing in mouse tumor model using CT2A gliomas in C57B16 mice using a variety of treatments including a combined poliovirus and checkpoint inhibitor treatment analogous to the invention; both the mice and the CT2A cells express the human poliovirus receptor CD155. Results (tumor volume over time) with the following experimental treatments are shown in the top panel: FIG. 4A, Group I: DMEM (vehicle to control for virus)+IgG (to control for anti-PD1); FIG. 4B, Group II: single intra-tumoral injection of PVSRIPO+IgG; FIG. 4C, Group III: single intra-tumoral injection of DMEM+anti-PD1; FIG. 4D, Group IV: single intra-tumoral injection of PVSRIPO (“mRIPO”)+anti-PD1. Anti-PD1 was given in three installments (days 3, 6, 9) by intraperitoneal injection. The three lower panels show tumor responses (tumor volume over time) in individual mice (each line a different mouse) in the treatment groups II-IV.

FIG. 5A-FIG. 5B show the results of treatment of mice with PVSRIPO (mRIPO) in combination with anti-PD1 or anti-PDL1 checkpoint inhibitor antibodies limits the growth in the E0771 orthotopic immunocompetent murine model of breast cancer. Mice were implanted in the mammary fatpad with 10⁶ E0771-CD155 tumor cells. PBS or mRIPO (5×10⁷ pfu) was injected into the tumors when they reached ˜100 mm³. Anti-PD1 (FIG. 5A)/anti-PDL1 (FIG. 5B) was injected intraperitoneally (250 μg in 200 μL PBS) the day of mRIPO injection and then every 2-3 days 4 times. Tumor growth was monitored over time. As shown in FIG. 5A, both mRIPO and anti-PD1 antibody were able to control tumor volume s compared to PBS, but the combination of mRIPO and anti-PD1 was significantly better. As shown in FIG. 5B, similar results were obtained using anti-PDL-1, where either mRIPO or anti-PDL1 alone were able to control tumor growth better than PBS control, but the combination of mRIPO and anti-PDL1 resulted in decreased tumor growth.

FIG. 6A-FIG. 6B show the results of various treatments of C57BL/6-CD155 transgenic mice orthotopically implanted with 5×10⁵ E0771-CD155 cells. FIG. 6A is a graph of tumor volume over the number of days post tumor implant of mice receiving (i) neoadjuvant therapy (mRIPO followed by surgery (—★—), (ii) receiving treatment with PBS followed by surgery (—♦—), (iii) receiving no surgery and treatment with mRIPO (—▪—), and (iv) receiving no surgery and treatment with PBS (—•—). Significance is denoted by p values: ★, P≤0.05; ★★, P≤0.01; ★★★, P≤0.001. FIG. 6B is a graph of tumor volume over the number of days post tumor re-challenge of mice treated with mRIPO followed by surgery (—★—) compared to mice treated with PBS followed by surgery (—♦—).

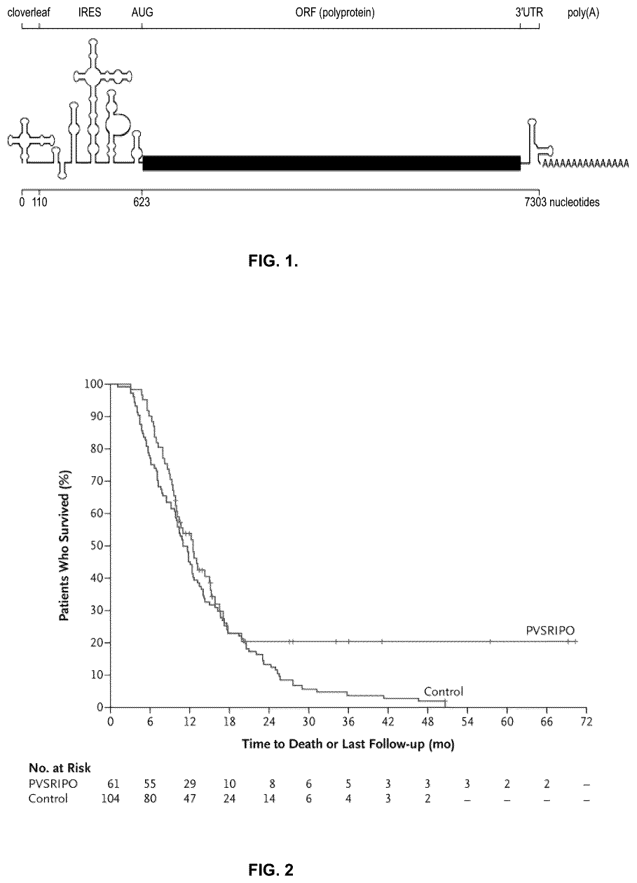

### DETAILED DESCRIPTION OF THE INVENTION

While neoadjuvant chemotherapy of cancer has been applied for several years, neoadjuvant immunotherapy of cancer is still a developing medical application. The inventors have developed neoadjuvant immunotherapy (also referred to herein as neoadjuvant therapy) in which one or more immunotherapeutic agents, comprising an oncolytic chimeric poliovirus construct or a combination comprising an oncolytic chimeric poliovirus construct and an immune checkpoint inhibitor, is administered to a human having tumor. Following administration of the one or more immunotherapeutic agents, the tumor treated by the one or more immunotherapeutic agents is then reduced (e.g., resected by surgery, or reduced in size and/or amount by radiation therapy). Optionally, the individual may then receive maintenance therapy comprising the one or more immunotherapeutic agents. Unexpectedly, one or more therapeutic benefits are observed for individuals treated with the neoadjuvant immunotherapy comprising an oncolytic chimeric poliovirus construct (e.g., PVSRIPO as described in U.S. Pat. No. 6,264,940, which is incorporated herein by reference in its entirety), or a combination of an oncolytic chimeric poliovirus construct and an immune checkpoint inhibitor. These therapeutic benefits were not apparent at the time of the invention. For example, at the time of the invention it was known that pathological complete response rates observed from use of neoadjuvant therapy does not always translate into improved survival, as has been observed in some patients with breast cancer following neoadjuvant therapy. Additionally, tumors with a low mutational burden are most responsive to treatment by the oncolytic chimeric poliovirus construct PVSRIPO; whereas (and in contrast) responsiveness to immune checkpoint blockade from treatment with an immune checkpoint inhibitor are predominately by tumors with high mutational burden. Also, PVSRIPO has been used in clinical trials in an adjuvant setting; i.e., where the tumor is not resected after treatment with PVSRIPO. In the adjuvant setting, tumor cells are infected by PVSRIPO, more infectious virus is produced, infected tumor cells are lysed by the virus, newly produced infectious virus is released which can then infect additional tumor cells of the tumor, and the cycle is repeated. Newly produced virus can also further stimulate dendritic cells in inducing an antitumor immune response. This repeated cycle of tumor infection and lysis, and further stimulation of the immune response is limited in neoadjuvant therapy, since tumor burden is reduced after the administration of PVSRIPO and an immune checkpoint inhibitor. Thus, durability of a resultant antitumor response, as observed by increased survival rates or other observed therapeutic benefits, would be unexpected with this neoadjuvant immunotherapy.

In the methods of the invention, any technique for directly administering an oncolytic chimeric poliovirus construct to the tumor may be used. Direct administration does not rely on the blood vasculature to access the tumor. The preparation may be painted on the surface of the tumor, injected into the tumor, instilled in or at the tumor site during surgery, infused into the tumor via a catheter, etc. One particular technique for treating brain cancers which may be used is convection enhanced delivery. The oncolytic chimeric poliovirus construct is a recombinant or genetically engineered poliovirus in which the native poliovirus IRES is at least partially exchanged with the IRES of other picornaviruses, such as human rhinovirus 2. The poliovirus is generally a Sabin poliovirus and suitably a Sabin type I strain of poliovirus. Thus in the 5′ untranslated region (UTR) of the engineered oncolytic chimeric poliovirus constructs described herein, the 5′ cloverleaf of the native poliovirus is included and the native IRES of the poliovirus is at least partially replaced with an IRES from human rhinovirus 2 and the rest of the native or wild-type poliovirus open reading frame is kept intact.

Immune checkpoint inhibitors which may be used according to the invention are any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, and/or anti-TIM-3 antibody. Approved checkpoint inhibitors in the U.S. include atezolizumab, ipimilumab, pembrolizumab, and nivolumab, and tislelizumab. The inhibitor need not be an antibody, but can be a small molecule or other polymer. If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and the B-7 family of ligands. Combinations of inhibitors for a single target immune checkpoint or different inhibitors for different immune checkpoints may be used. Additionally, CSF-1R blockade may be used in combination or as an alternative to immune checkpoint inhibitor(s), to ensure generation of potent and sustained immunity that effectively eliminates distant metastases and recurrent tumors. Antibodies specific for CSF-1R or drugs that inhibit or blockade CSF-1R may be used for this purpose, including but not limited to imactuzumab and AMG820.

In a method of neoadjuvant therapy, one or more immunotherapeutic agents (a therapeutically effective amount of an oncolytic chimeric poliovirus construct, or of an immune checkpoint inhibitor and an oncolytic chimeric poliovirus construct) is administered prior to an individual undergoing treatment by surgery or radiation to reduce the amount of tumor in the individual. Typically, wherein the neoadjuvant therapy comprises two immunotherapeutic agents, the two agents will be administered within days of each other. For example, an immune checkpoint inhibitor is administered followed by administration of oncolytic chimeric poliovirus construct at 30, 28, 21, 14, 10, 9, 8, 7, 6, 5, 4, 3, 2, or 1 day(s) after administration of the immune checkpoint inhibitor. Alternatively, it may be advantageous to administer the oncolytic chimeric poliovirus construct prior to administration of an immune checkpoint inhibitor, wherein the immune checkpoint inhibitor is then administered to the individual within several days or weeks (e.g., at 30, 28, 21, 14, 10, 9, 8, 7, 6, 5, 4, 3, 2, or 1 day(s)) after administration of the oncolytic chimeric poliovirus construct. Priming of a cytotoxic T lymphocyte response by the oncolytic chimeric poliovirus construct may take from about 5 to about 14 days. Administration of the immune checkpoint inhibitor may beneficially be commenced before, during, or after such priming period. For example, in one aspect, the immune checkpoint inhibitor is administered 14 days after administration of the oncolytic chimeric poliovirus construct, and after about 1 week to about 3 weeks following administration of the immune checkpoint inhibitor, the individual is then treated to reduce tumor burden (e.g., by surgery or radiation therapy). Typically, wherein the neoadjuvant therapy comprises administration of oncolytic chimeric poliovirus, about 1 week to about 3 weeks later after receiving the oncolytic chimeric poliovirus construct, the individual is then treated to reduce tumor burden (e.g., by surgery or radiation therapy). Optionally, following reduction of tumor burden, the individual may receive maintenance therapy with an immune checkpoint inhibitor which comprised periodic (e.g., about every 1 week to 3 weeks) administration of a therapeutically effective amount of an immune checkpoint inhibitor, and/or may be administered in combination with the oncolytic chimeric poliovirus construct should the tumor recur.

A therapeutically effective amount of an immunotherapeutic agent comprising the oncolytic chimeric poliovirus construct or the immune checkpoint inhibitor is an amount effective to cause a therapeutic benefit to an individual receiving the immunotherapeutic agent. Such an effective amount may vary according to characteristics of the individual, including health status, gender, size (e.g., body weight), age, cancer type, cancer stage, route of administration, tolerance to therapy, toxicity or side effects, and other factors that a skilled medical practitioner would take into account when establishing appropriate treatment dosing and regimen. For example, a therapeutically effective amount of an oncolytic chimeric poliovirus construct may range from about 1×10⁸ tissue culture infectious dose (TCID) to about 5×10⁶ TCID. A therapeutically effective amount of an immune checkpoint inhibitor may range from about 0.5 mg/kg of body weight to about 5 mg/kg of body weight; from about 1 mg/kg of body weight to about 5 mg/kg of body weight; from about 1 mg/kg of body weight to about 3 mg/kg of body weight; from about 500 mg to about 1500 mg, or lesser or greater amounts as determined by a medical practitioner.

An immune checkpoint inhibitor may be administered by any appropriate means known in the art for the particular inhibitor. These include intravenous, oral, intraperitoneal, sublingual, intrathecal, intracavitary, intramuscularly, intratumorally, and subcutaneously. Optionally, the immune checkpoint inhibitor may be administered in combination with an oncolytic chimeric poliovirus construct.

Any human tumor can be treated by this method of neoadjuvant therapy, including both pediatric and adult tumors. The tumor may be in any organ, for example, brain, prostate, breast, lung, colon, and skin. Various types of tumors may be treated, including, for example, glioblastoma, medulloblastomas, carcinoma, adenocarcinoma, etc. Other examples of tumors include, adrenocortical carcinoma, anal cancer, appendix cancer, grade I (anaplastic) astrocytoma, grade II astrocytoma, grade III astrocytoma, grade IV astrocytoma, atypical teratoid/rhabdoid tumor of the central nervous system, basal cell carcinoma, bladder cancer, breast sarcoma, bronchial cancer, bronchoalveolar carcinoma, cervical cancer, craniopharyngioma, endometrial cancer, endometrial uterine cancer, ependymoblastoma, ependymoma, esophageal cancer, esthesioneuroblastoma, Ewing's sarcoma, extracranial germ cell tumor, extragonadal germ cell tumor, extrahepatic bile duct cancer, fibrous histiocytoma, gall bladder cancer, gastric cancer, gastrointestinal carcinoid tumor, gastrointestinal stromal tumor, gestational trophoblastic tumor, gestational trophoblastic tumor, glioma, head and neck cancer, hepatocellular cancer, Hilar cholangiocarcinoma, hypopharyngeal cancer, intraocular melanoma, islet cell tumor, Kaposi sarcoma, Langerhans cell histiocytosis, large-cell undifferentiated lung carcinoma, laryngeal cancer, lip cancer, lung adenocarcinoma, malignant fibrous histiocytoma, medulloepithelioma, melanoma, Merkel cell carcinoma, mesothelioma, endocrine neoplasia, nasal cavity cancer, nasopharyngeal cancer, neuroblastoma, oral cancer, oropharyngeal cancer, osteosarcoma, ovarian clear cell carcinoma, ovarian epithelial cancer, ovarian germ cell tumor, pancreatic cancer, papillomatosis, paranasal sinus cancer, parathyroid cancer, penile cancer, pharyngeal cancer, pineal parenchymal tumor, pineoblastoma, pituitary tumor, pleuropulmonary blastoma, renal cell cancer, respiratory tract cancer with chromosome 15 changes, retinoblastoma, rhabdomyosarcoma, salivary gland cancer, small cell lung cancer, small intestine cancer, soft tissue sarcoma, squamous cell carcinoma, squamous non-small cell lung cancer, squamous neck cancer, supratentorial primitive neuroectodermal tumor, supratentorial primitive neuroectodermal tumor, testicular cancer, throat cancer, thymic carcinoma, thymoma, thyroid cancer, cancer of the renal pelvis, urethral cancer, uterine sarcoma, vaginal cancer, vulvar cancer, and Wilms tumor.

Optionally, individuals having tumor may be stratified for treatment on the basis of NECL5 (CD155, poliovirus receptor) expression by the individual's tumor prior to treatment according to the methods described herein. This can be assayed at the RNA or protein level, using probes, primers, or antibodies, for example. The NECL5 expression may guide the decision to treat or not treat with the oncolytic chimeric poliovirus construct. The NECL5 expression may also be used to guide the aggressiveness of the treatment, including the dose, frequency, and duration of treatments. Antibodies to NECL5 (CD155) are commercially available and may be used. NECL5 RNA expression can also be assayed, using methods known in the art.

In addition to neoadjuvant therapy comprising administering oncolytic chimeric poliovirus construct and one or more immune checkpoint inhibitors followed by surgical removal of the tumor or surgical reduction of the tumor, treatment of the individual may comprise one or more of chemotherapy, biological therapy, and radiotherapy. These modalities may be current standard of care for treatment of certain human tumors. The neoadjuvant therapy may be administered before, during, or after the standard of care for treating the tumor. For example, PVSRIPO and immune checkpoint inhibitor combination comprising neoadjuvant therapy may be administered after failure of the standard of care. When a combination of immunotherapeutic agents is specified, each agent may be administered separately in time as two separate agents within a single combination regimen. Alternatively, the two (or more) agents may be administered in admixture.

Kits may comprise, in a single divided or undivided container, both the oncolytic chimeric poliovirus construct, e.g., PVSRIPO, as well as an immune checkpoint inhibitor. The two agents may be in separate vessels, or in a single vessel in admixture. Instructions for administration may be included. Optionally, included as a component of the kit is an antibody and reagents or PCR primers for testing NECL5 expression by an individual's tumor.

Applicants have developed methods for production of oncolytic chimeric poliovirus construct and methods to test for genetic stability and homogeneity. Any suitable method for production and testing for genetic stability can be used. For example, methods for assessing stability include testing for the inability to grow at 39.5 degrees C., bulk sequencing to determine the presence or absence of mutations, and testing for primate neurovirulence.

Multiple mechanisms may contribute to the efficacy of the oncolytic chimeric poliovirus construct, PVSRIPO, in inducing an antitumor immune response, including infection and lysis of cancer cells, infection and activation of antigen presenting cells, and recruitment and activation of immune cells for targeting cancer cells. Hence, treatment of tumor with PVSRIPO comprises immunotherapy, in addition to direct killing of tumor by the virus.

While the terms used in the description of the invention are believed to be well understood by one of ordinary skill in oncology and medicine, definitions, where provided herein, are set forth to facilitate description of the invention, and to provide illustrative examples for use of the terms.

As used herein, the terms “a”, “an”, and “the” mean “one or more”, unless the singular is expressly specified (e.g., singular is expressly specified, for example, in the phrase “a single agent”).

As used herein, the term “pharmaceutically acceptable carrier” means any compound or composition or carrier medium useful in any one or more of administration, delivery, storage, stability of a composition or combination described herein. These carriers are known in the art to include, but are not limited to, a diluent, water, saline, suitable vehicle (e.g., liposome, microparticle, nanoparticle, emulsion, capsule), buffer, tracking agents, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbant, flavor agent, and the like as broadly known in the pharmaceutical art.

Treating cancer or treating an individual with a tumor includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a individual as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

A “therapeutically effective amount” or an effective amount as used herein means the amount of a composition that, when administered to a subject for treating a tumor is sufficient to effect a treatment (as defined above). The therapeutically effective amount will vary depending on the formulation or composition, the tumor type and its severity and the age, weight, physical condition and responsiveness of the subject to be treated.

“Neoadjuvant therapy” is used herein to refer to therapy given to an individual having tumor before the individual undergoes reduction of tumor burden, such as surgery to remove or reduce the amount of tumor, or radiation therapy to reduce the amount of tumor. Surgery can involve whole resection or partial resection of tumor. Neoadjuvant therapy may result in a reduction of tumor burden which may facilitate subsequent resection.

“Adjuvant therapy” is used herein to refer to therapy given after surgery for resection tumor.

“Maintenance therapy” is used herein to refer to therapeutic regimen that is given to reduce the likelihood of disease progression or recurrence. Maintenance therapy can be provided for any length of time depending on assessment of clinical parameters for assessing response to therapy.

“Survival” is used herein to refer to an individual remaining alive after treatment, and includes overall survival, and disease-free survival. Survival is typically measured by the Kaplan-Meier method. Disease-free survival refers to a treated individual remaining alive without evidence of recurrence of cancer. Overall survival refers to an individual remaining alive for a defined period of time.

The above disclosure generally describes the present invention. A more complete understanding can be obtained by reference to the following specific examples, which are provided herein for purposes of illustration only, and are not intended to limit the scope of the invention.

### Example 1

A Phase I clinical trial was conducted in individuals with tumor using PVSRIPO alone. The tumor was recurrent glioblastoma (GBM), and PVSRIPO was administered after tumor resection (adjuvant therapy). A number of dosages were tested, including 1×10⁸ tissue culture infectious dose (TCID), 5×10⁷ TCID, and 1×10⁷ TCID. PVSRIPO (“PVSRIPO DL 1-5”, FIG. 2, Table 1) was delivered directly into the tumor. Convection-enhanced delivery was used to infuse PVSRIPO intratumorally. An implanted catheter was used to infuse PVSRIPO at a delivery rate of 500 μL/hr, with 3 mL being the total amount of the inoculum delivered to the individual. The results of the Phase I trial are summarized in Table 1, and in FIG. 2 (followed up to Mar. 20, 2018), wherein individuals treated with PVSRIPO are compared to historical controls. As shown in Table 1 and FIG. 2, overall survival for individuals treated with PVSRIPOP is significantly improved, particularly at 2 years and beyond, as compared to historical controls.

TABLE 1
PVSRIPO dose escalation in patients vs Historical Control: Overall survival

| | | | 12-month | 24-month | 36-month | 48-month | 60-month |
|---|---|---|---|---|---|---|---|
| | | # | survival | survival | survival | survival | survival |
| Group | Total | Failed | (95% CI) | (95% CI) | (95% CI) | (95% CI) | (95% CI) |
| PVSRIPO | 15 | 12 | 60.0% | 20.0% | 20.0% | 20.0% | 20.0% |
| DL 1-5 | | | (31.8%, 79.7%) | (4.9%, 42.4%) | (4.9%, 42.4%) | (4.9%, 42.4%) | (4.9%, 42.4%) |
| Historical | 104 | 103 | 45.2% | 13.5% | 3.8% | 1.9% | 0% |
| controls | | | (35.5%, 54.4%) | (7.8%, 20.7%) | (1.3%, 8.8%) | (0.4%, 6.1%) | |

### Example 2

The mechanism of immune checkpoint inhibitors is to release cytotoxic T cell function from events instigated by tumors that block their effector functions. Tumors engage a system of naturally existing ‘brakes’ that control cytotoxic T cells. To the tumor, this has the advantage of limiting the potential for the immune system to attack tumors that express mutant proteins and, therefore, represent a foreign signature. Immune checkpoint inhibitors reverse this tumor mechanism and release immune function. PVSRIPO elicits an immune response that induces cytotoxic T cells (CTL) to attack tumors. Thus, combination of PVSRIPO with immune checkpoint inhibitors enhances the therapeutic effect. As shown below, PVSRIPO, indeed, works to treat tumors by inducing CTL responses.

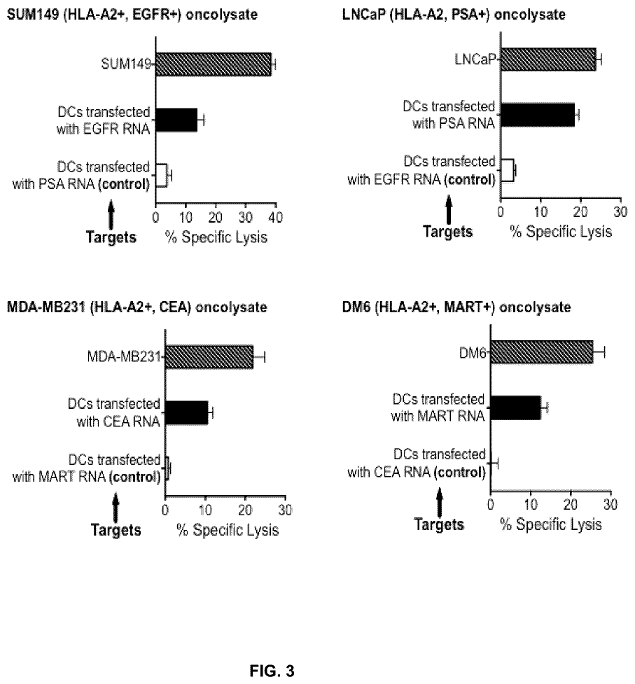

Melanoma, breast, brain tumor, prostate cancer cells were contacted and infected with PVSRIPO in culture, and supernatants from dying/dead cells in the cultures were collected. The supernatants from the infected tumor cells were used to expose dendritic cells (a population of immune cells that is responsible for communicating with CTLs and coordinating their activation) isolated from human subjects. As a consequence, the dendritic cells exhibited powerful signs of pro-inflammatory activation (i.e., the virus infection of the tumor cells produced soluble factors that promoted the CTL activation functions of dendritic cells; and virus released from infected tumor cells activated the dendritic cells). The activated dendritic cells were then co-cultivated with T cells (including CTLs) from the same human subject that donated the dendritic cells. The co-cultured T cells (including CTLs) were then co-cultivated with uninfected tumor cells from the same lines used for the infection step. As shown in FIG. 3, observed was a high-level of cytotoxicity of the activated CTLs against the tumor cells.

This experiment, in vitro, exemplifies what is believed to occur in individuals with tumor who are treated with PVSRIPO: virus infection elicits a series of events that ultimately leads to the generation of a CTL response against the tumor. This series of events can be enhanced synergistically with immune checkpoint inhibitors. One of the natural existing ‘brakes’ on T cell function (immune checkpoints) is the PD1-PD-L1 link. Dendritic cells in tumor often are induced to express PD-L1, which then binds to PD1 on T cells to inhibit activation of the T cells. Demonstrated is that dendritic cells exposed to PVSRIPO/PVSRIPO-tumor lysate increase PD-L1 expression. PD-1 or PD-L1 inhibitors, paradigmatic checkpoint inhibitors, prevent this effect and increase CTL activation by PVSRIPO oncolysis.

In this experiment, confluent 10 cm dishes of Sum149, MDAMB231, LNCaP, or DM6 cells were infected with mock (DMEM) or PVSRIPO (MOI 0.1) in AIMV medium for 48 hours. Supernatants were collected and cell debris was removed by centrifugation. Frozen PBMCs were thawed, washed in PBS and resuspended at 2×10⁸ cells in 30 ml AIM-V media in T-150 tissue culture flasks. Cells were incubated for 1 hour at 37° C. The non-adherent cells were harvested by rocking the flask from side to side to dislodge them. The adherent cells were replenished with 30 ml AIM-V supplemented with 800 U/ml human GM-CSF and 500 U/ml human IL-4, then incubated at 37° C. DCs were harvested on day 6, by collecting all non-adherent cells, followed by a cold PBS wash. Cells that were still adherent were dissociated with cell dissociation buffer. DCs were washed in AIMV medium, counted and seeded in 35 mm dishes at 1×10⁶ cells per dish. Supernatant from onco-lysate was added to DC cultures and incubated for 24 hours. Supernatant was then removed and DCs were washed in AIMV medium. PBMCs were thawed and resuspended in PBS and treated with DNase I at 200 U/ml for 20 minutes at 37° C. DNase I-treated PBMCs were incubated for 1 hour at 37° C., Non-adherent cells were harvested and stimulated with DCs loaded with poliovirus-induced tumor lysate at a responder cell to stimulator DC ratio of 10:1 in the presence of 25 ng/ml IL-7. All stimulations were done in RPMI 1640 with 10% FCS, 2 mM L-glutamine, 20 mM HEPES, 1 mM sodium pyruvate, 0.1 mM MEM non-essential amino acids, 100 IU/ml penicillin, 100 μg/ml streptomycin and 5×10⁻⁵ M ß-mercaptoethanol (CTL stimulation medium). The responder T-cell concentration was 2×10⁶ cells/ml. IL-2 was added at 100 U/ml on day 3 and every 4-5 days for 12-14 days. T cells were maintained at 1-2×10⁶ cells/ml in CTL stimulation medium. T cells were harvested on day 12-14, counted and used as effector T cells in a europium-release CTL assay. Autologous DCs transfected with tumor antigen-encoding mRNA were used as targets as controls. For DC target controls, mRNA-electroporated target cells (as designated in FIG. 2) were harvested, washed to remove all traces of media and labeled with europium (Eu). Alternatively, original target cells (Sum149, MDAMB231, LNCaP, or DM6) were labeled with Eu. The Eu-labeling buffer (1 ml per target) contained 1 ml HEPES buffer (50 mM HEPES, 93 mM NaCl, 5 mM KCl, 2 mM MgCl₂, pH 7.4), 10 μl Eu (10 mM EuCl₃·6H₂O in 0.01 N HCl), 5 μl DTPA (100 mM diethylenetriamine pentaacetate in HEPES buffer) and 4 μl DS (1% dextran-sulfate). 5×10⁶ target cells were resuspended in 1 ml of the europium-labeling buffer very gently and incubated on ice for 20 minutes. 30 μl of CaCl2 solution (100 mM) was then added to the labeled cells, mixed and the cells were incubated for another 5 minutes on ice. 30 ml of Repair buffer (HEPES buffer with 10 mM glucose, 2 mM CaCl₂)) was added to the cells and the cells were centrifuged at 1000 rpm for 10 minutes. Cells were counted and 5×10⁶ cells were washed 4 times with Repair buffer. After the final wash the cells were resuspended in CTL stimulation medium without penicillin-streptomycin at 10⁵ cells/ml. Ten thousand europium-labeled targets (T) and serial dilutions of effector cells (E) at varying E:T ratios were incubated in 200 μl of CTL stimulation medium with no penicillin-streptomycin in 96-well V-bottom plates. The plates were centrifuged at 500×g for 3 minutes and incubated at 37° C. for 4 hours. 50 μl of the supernatant was harvested and added to 150 μl of enhancement solution in 96-well flat-bottom plates and europium release was measured by time resolved fluorescence using the VICTOR3 Multilabel Counter (Perkin-Elmer). Specific cytotoxic activity was determined using the formula: % specific release=[(experimental release−spontaneous release)/(total release−spontaneous release)]×100. Spontaneous release of the target cells was less than 25% of total release by detergent. Spontaneous release of the target cells was determined by incubating the target cells in medium without T cells. All assays were done in triplicate, bars represent average % lysis and error bars denote SEM.

### Example 3

PVSRIPO antitumor efficacy may be aided by the virus' ability to elicit strongly immunogenic type 1 interferon (IFN) responses in infected tumor cells and in infected antigen-presenting cells (dendritic cells, macrophages, microglia). However, although type 1 IFN responses are highly desirable as mediators of immunotherapy, they also engage known immune checkpoints that can dampen the anti-neoplastic immune response elicited by PVSRIPO, e.g., PD-L1. Therefore, efforts to maximize PVSRIPO immunotherapy by combination with immune checkpoint blockade may be investigated.

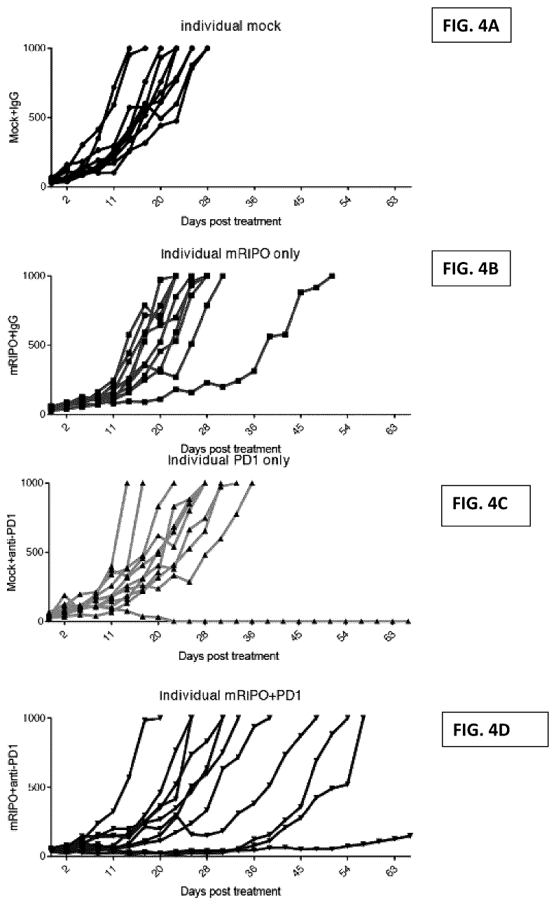

In this experiment, CT2A gliomas were implanted subcutaneously in C57B16 mice transgenic for the poliovirus receptor CD155. The CT2A cells used to initiate tumors were previously transduced with CD155 (to enable PVSRIPO infection analogous to human cells). Four groups of tumor-bearing animals (n=10) were treated as follows: Group I: DMEM (vehicle to control for virus)+IgG (to control for anti-PD1); Group II: single intra-tumoral injection of PVSRIPO+IgG; Group III: single intra-tumoral injection of DMEM+anti-PD1; Group IV: single intra-tumoral injection of PVSRIPO+anti-PD1. Anti-PD1 was given in three installments (days 3, 6, 9) by intraperitoneal injection. Results are shown in FIGS. 4A-4D.

Both PVSRIPO and anti-PD1 had significant anti-tumor effects individually (FIG. 4B; FIG. 4C). The combination of the two agents had added therapeutic effects, suggesting mechanistic synergy (FIG. 4D). Importantly, durable tumor remission (indicated by flat-lining of the tumor response curves at very low tumor volumes) was only achieved with the combination treatment.

### Example 4

This example provides another illustration of the combination of an oncolytic virus, oncolytic chimeric poliovirus PVSRIPO, with an immune checkpoint inhibitor in mediating significant anti-tumor effects. In these studies, used as a standard experimental model for breast cancer was the E0771 orthotopic breast tumor model. This model is representative of triple negative breast cancer (TNBC). The murine tumor cell line E0771 was transfected with human CD155, the poliovirus receptor, to make the cells (“E0771-CD155”) susceptible to infection by oncolytic poliovirus, PVSRIPO. To ensure replication in mouse tumor cell lines, PVSRIPO was passaged in mouse tumor cell lines to generate mouse PVSRIPO (mRIPO). All studies were conducted in C57BL/6-CD155 transgenic mice. Mice were implanted in the mammary fatpad with 10⁶ E0771-CD155 tumor cells. PBS or mRIPO (5×10⁷ pfu) was injected into the tumors when they reached 70-100 mm³. Immune checkpoint inhibitor anti-PDL1 antibody or anti-PD1 antibody (250 μg in 200 μL PBS) was injected intraperitoneally on the day of mRIPO injection, and then every 2-3 days for a total of four injections of immune checkpoint inhibitor. Tumor growth was then monitored over time.

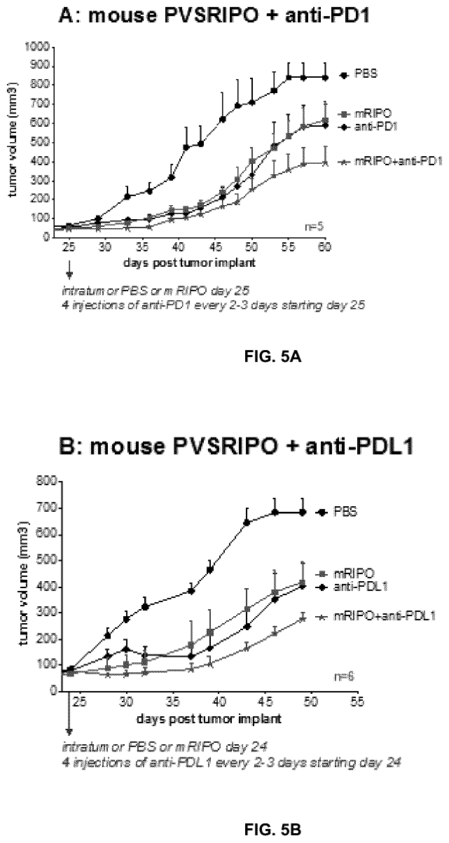

Tested was whether by blocking the PD1/PDL1 pathway using an antibody that targets PD1 or PDL1 in combination with mRIPO is superior at controlling tumor growth as compared to each as a monotherapy (mRIPO alone, anti-PD1antibody alone, or anti-PDL1 antibody alone). As shown in FIGS. 5A & 5B, oncolytic poliovirus alone (mRIPO, ▪), anti-PD1 antibody (anti-PD1, FIG. 5A-♦), or anti-PDL1 antibody alone (anti-PDL1, FIG. 5B-♦), and combination therapy mRIPO plus anti-PD1/PDL1 significantly inhibited tumor growth compared to PBS control. There were no significant differences in tumor growth inhibition between mRIPO and anti-PD1 (FIG. 5A) or anti-PDL1 (FIG. 5B) monotherapies throughout the study. Combination of mRIPO with anti-PD1 or anti-PDL1 was more effective than each monotherapy alone at controlling tumor growth toward the end of the study (not statistically significant). This preliminary experiment indicates that the combination of PVSRIPO with anti-PD1/PDL1 therapy trended towards synergistic improvement in tumor growth inhibition in the murine orthotopic immunocompetent breast cancer model.

### Example 5

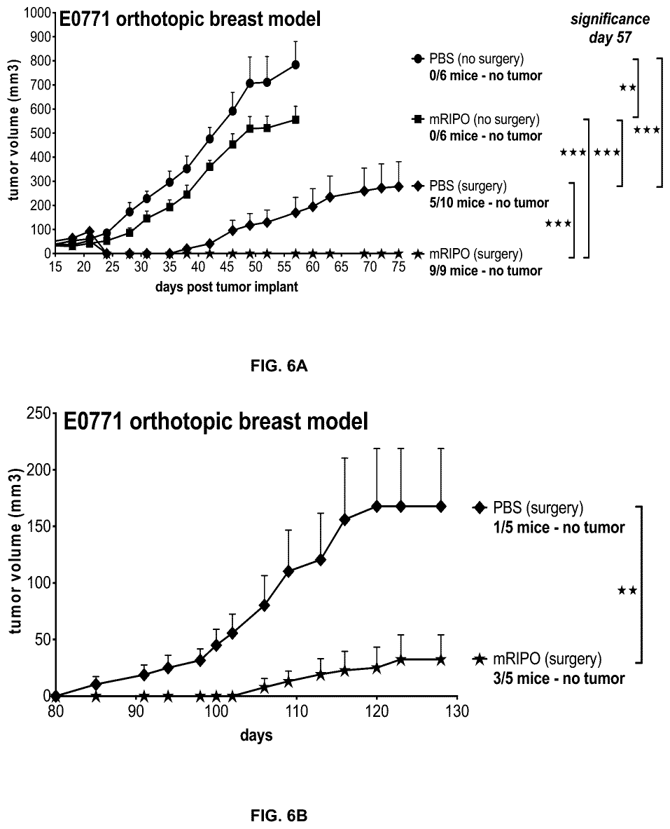

Provided is neoadjuvant therapy using one or more immunotherapeutic agents. In this example, C57BL/6-CD155 transgenic mice were orthotopically implanted with 5×10⁵ E0771-CD155 cells. Fifteen days following tumor implant, mice were either treated with mRIPO or PBS (each injected intratumorally once tumors reached ˜50 mm³ in size), followed by either surgery at day 22 following tumor implant, or no surgery. As shown in FIG. 6A, in the group receiving neoadjuvant therapy (mRIPO followed by surgery; FIG. 6A, —★—) 9 out of 9 treated were tumor-free, as compared to 5/10 mice who received treatment with PBS followed by surgery (FIG. 6A, —♦—). In contrast, all mice in the no surgery groups (whether received PBS or mRIPO) developed tumors, where treatment with mRIPO (FIG. 6A, —▪—) being more effective at control of tumor growth control as compared to treatment with PBS (FIG. 6A, —•—). Five mice from the group treated with PBS followed by surgery and five mice treated with mRIPO followed by surgery were re-challenged with parent E0771 cells on day 80 following tumor implantation. As shown in FIG. 6B, on day 130 following tumor implantation, 3 of the 5 mice receiving the neoadjuvant therapy (mice treated with mRIPO followed by surgery; FIG. 6B; —★—) compared to 1 out of 5 mice in the PBS-treated group (FIG. 6B; —♦—) had no tumors.

### Example 6

Provided is a method of treating an individual having tumor, comprising administering to the individual a therapeutically effective amount of an immune checkpoint inhibitor and a therapeutically effective amount of an oncolytic chimeric poliovirus construct prior to surgical resection of tumor, performing surgery to resect the tumor, wherein after resection of tumor administered to the individual is immune check point inhibitor. To illustrate this method of neoadjuvant therapy, approximately 1 week before administration of PVSRIPO, the individual having tumor that has not been resected receives a commercially available poliovirus immunization booster, and treatment is initiated by administering PVSRIPO to the individual. For example, PVSRIPO may be administered intratumorally. In this example, several (from about 7 to about 14) days after treatment with PVSRIPO, anti-PD-1 antibody is then administered to the individual. The anti-PD1 antibody may be administered intravenously. One to three weeks post-administration of the anti-PD1 antibody, the individual is treated to reduce tumor burden (e.g., the tumor is surgically resected). Optionally, following reduction of tumor burden, the individual may receive maintenance therapy comprising administering the immune checkpoint inhibiter as medically warranted, anti-PD-1 antibody may be administered every 2 weeks for 4 months, then every 4 weeks for up to 2 years.

We claim:

1. A method of treating an individual having a tumor, the method comprising:
a) administering to the individual a therapeutically effective amount of an immune checkpoint inhibitor and a therapeutically effective amount of an oncolytic chimeric poliovirus construct prior to surgical resection of tumor,
b) subsequently performing surgery to resect the tumor,
c) after resection of the tumor, administering to the individual a therapeutically effective amount of an immune check point inhibitor; and wherein the oncolytic chimeric poliovirus construct optionally comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame.

2. A method for neoadjuvant immunotherapy of cancer comprising:
a) administering one or more immunotherapeutic agents in a therapeutically effective amount to an individual having a tumor, wherein the one or more immunotherapeutic agents comprise an oncolytic chimeric poliovirus construct, or an oncolytic chimeric poliovirus construct and an immune checkpoint inhibitor;
b) subsequent to receiving the one or more immunotherapeutic agents, treating the individual with anti-cancer therapy effective to reduce tumor burden in the individual.

3. The method of claim 2, wherein the anti-cancer therapy is selected from the group consisting of surgery, radiation therapy or a combination thereof.

4. The method of any one of claim 2 or 3, wherein the oncolytic chimeric poliovirus construct comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame.

5. The method of claim 2, wherein only one immunotherapeutic agent is administered to the individual having the tumor and prior to the individual receiving anti-cancer therapy to reduce tumor burden, and wherein the immunotherapeutic agent comprises a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame.

6. The method of claim 2, wherein subsequent to receiving anti-cancer therapy to reduce tumor burden, the method further comprises the individual receiving maintenance therapy comprising one or more of the oncolytic chimeric poliovirus construct, or the immune checkpoint inhibitor.

7. The method of any one of claims 1-6, wherein the oncolytic chimeric poliovirus construct further comprises a pharmaceutically acceptable carrier.

8. The method of any one of claims 1-7, wherein the immune checkpoint inhibitor further comprises a pharmaceutically acceptable carrier.

9. The method of any one of claims 1-8, wherein the tumor is selected from the group consisting of a brain tumor, renal cell carcinoma, prostate tumor, bladder tumor, esophageal tumor, stomach tumor, pancreatic tumor, colorectal tumor, liver tumor, gall bladder tumor, breast tumor, lung tumor, head and neck tumor, skin tumor, melanoma, and sarcoma.

10. The method of claim 1 or claim 2, wherein the tumor expresses NECL5 (nectin-like protein 5).

11. The method of any one of claims 1-9, wherein the tumor expresses NECL5 (nectin-like protein 5).

12. The method of any one of claims 1-11, wherein the oncolytic chimeric poliovirus construct is administered directly to the tumor.

13. The method of claim 1 or claim 2, wherein prior to administering the oncolytic chimeric poliovirus construct to the individual, the method comprises the step of testing the individual's tumor to ascertain expression of NECL5.

14. The method of any one of claims 1-12, wherein prior to administering the oncolytic chimeric poliovirus construct to the individual, the method comprises the step of testing the individual's tumor to ascertain expression of NECL5.

15. The method of claim 1 or claim 2, wherein the immune checkpoint inhibitor is selected from the group consisting of an anti-PD-1 antibody, an anti-PDL-1 antibody, an anti-CTLA4 antibody, an anti-LAG-3 antibody, and an anti-TIM-3 antibody.

16. The method of any one of claims 1-14, wherein the immune checkpoint inhibitor is selected from the group consisting of an anti-PD-1 antibody, an anti-PDL-1 antibody, an anti-CTLA4 antibody, an anti-LAG-3 antibody, and an anti-TIM-3 antibody.

17. The method of claim 2, wherein an oncolytic chimeric poliovirus construct and an immune checkpoint inhibitor are administered to the individual having tumor.

18. The method of any one of claims 2-16, wherein both the oncolytic chimeric poliovirus construct and the immune checkpoint inhibitor are administered to the individual having the tumor.

19. The method of any one of claim 1, 17 or 18, wherein the oncolytic chimeric poliovirus construct is administered to the individual prior to the individual receiving an immune checkpoint inhibitor.

20. The method of any one of claim 1, 17 or 18, wherein the immune checkpoint inhibitor is administered to the individual prior to the individual receiving the oncolytic chimeric poliovirus construct.

21. The method of claim 1 or claim 2, further comprising administering a poliovirus immunization booster to the individual several days before administering the oncolytic chimeric poliovirus construct.

22. The method of any one of claims 1-20, further comprising administering a poliovirus immunization booster to the individual several days before administering the oncolytic chimeric poliovirus construct.

23. The method of claim 1 or claim 2, further comprising administering multiple doses of immune checkpoint inhibitor to the individual following resection or reduction of the tumor, wherein the doses are separated by days or weeks.

24. The method of any one of claims 1-22, further comprising administering multiple doses of immune checkpoint inhibitor to the individual following resection or reduction of the tumor, wherein the doses are separated by days or weeks.

25. A kit comprising an oncolytic chimeric poliovirus construct and an immune checkpoint inhibitor.

26. The kit of claim 25, wherein the oncolytic chimeric poliovirus construct is in a first container and the immune checkpoint inhibitor is in second container.

27. The kit of claim 25 or 26, further comprising reagents for resting NECL5 expression on a tumor cell sample.

28. The kit of claim 27, wherein the reagents include an antibody specific for NECL-5 or PCR primers specific for NECL-5.
